(19) United States
(12) Patent Application Publication
(10) Pub. No.: US 2023/0320697 A1
(43) Pub. Date: Oct. 12, 2023

(54) ULTRASOUND DIAGNOSTIC APPARATUS AND BEAM FORMING METHOD

(71) Applicant: FUJIFILM Healthcare Corporation, Kashiwa-shi (JP)

(72) Inventors: Motochika Shimizu, Chiba (JP); Kazuhiro Amino, Chiba (JP)

(21) Appl. No.: 18/127,147

(22) Filed: Mar. 28, 2023

(30) Foreign Application Priority Data
Apr. 7, 2022 (JP) 2022-064065

(51) Int. Cl. A61B 8/00; A61B 8/08

(52) U.S. Cl. CPC A61B 8/4488; A61B 8/54; A61B 8/5207

(57) ABSTRACT

A region of interest is defined in a living body. A plurality of transmission beams are simultaneously formed along a transmission center axis in such a manner that a plurality of transmission focal points are formed at a plurality of positions shallower than the region of interest on the transmission center axis. As a rest, a composite transmission beam is generated in the living body. After the composite transmission beam is formed, a reception beam set is generated.

### CROSS REFERENCE TO RELATED APPLICATION

This application claims priority to Japanese Patent Application No. 2022-064065 filed on Apr. 7, 2022, which is incorporated herein by reference in its entirety including the specification, claims, drawings, and abstract.

### TECHNICAL FIELD

The present disclosure relates to an ultrasound diagnostic apparatus and a beam forming method, and in particular, to formation of a transmission beam.

### BACKGROUND

Observation of a blood flow is useful for diagnosing cardiovascular diseases and medical treatment thereof. Given this backdrop, ultrasound diagnostic apparatuses have been utilized in the medical field.

A color doppler method has been known as a technique for observing a blood flow with the ultrasound diagnostic apparatus. The color doppler method is a method for measuring motion of an acoustic scatterer (mainly, red blood cells) by means of the doppler effect, which occurs when an ultrasound wave is reflected from the moving acoustic scatterer. Typically, in the color doppler method, a velocity distribution is calculated based on frame data acquired from a two-dimensional data acquisition region defined in a living body. In recent years, three-dimensional color doppler imaging performed by applying the color doppler method to volume data acquired from a three-dimensional data acquisition region within the living body is coming into widespread use.

In implementation of the color doppler method, it is necessary to secure a data acquisition rate (a frame rate or a volume rate) of a predetermined value or higher. For example, in the three-dimensional color doppler imaging, temporal changes in the flow of blood cannot be observed accurately when the volume rate is decreased to a low value of 5 to 6 Hz, for example. The frame rate or the volume rate can be improved by reducing the number of transmission beams formed to acquire data of one frame or one volume; i.e., by reducing a transmission beam density. In this case, however, image quality of a color doppler image will be deteriorated.

A parallel receive scheme is a scheme for simultaneously forming a plurality of spatially aligned reception beams with respect to one transmission beam. That is, in the parallel receive scheme, a set of reception beams is acquired by one transmitting and receiving step. When the parallel receive scheme is used, the reception beam density can be increased even in a situation where the transmission beam density cannot be increased due to limitations of the frame rate or the volume rate.

When an ultrasound image, such as a color doppler image, is generated using the parallel receive scheme, a blocky artifact is likely to occur in imaging. Specifically, because a sound pressure is gradually decreased as a distance from a center axis of a transmission beam (transmission center axis) in a beam scanning direction becomes greater, a difference in sound pressure can often arise between reception beams in each reception beam set and between two adjacent reception beam sets, which will be a cause of the occurrence of the blocky artifact. In particular, in a case where a spreading extent (full width at half maximum) of the transmission beam is small in the beam scanning direction in a situation where the transmission beam density is lowered, resulting in the presence of a low sound pressure part in the reception beam (for example, a low sound pressure part located outward of the full width at half maximum), the blocky artifact can conspicuously occur.

As a method for reducing the blocky artifact, there may be employed a technique of forming a transmission beam which has a focal point (single focal point) at a position shallower than a region of interest (ROI) specified by a user. The thus-formed transmission beam may be referred to as a near focus wide beam. The near focus wide beam has a divergent portion (a diffusing portion) in a region farther than the focal point. The divergent portion passes through the region of interest. Because the divergent portion has a relatively great full width at half maximum, the blocky artifact is less likely to occur when parallel reception is performed.

Meanwhile, it is necessary in terms of safety of the living body that a mechanical index (MI) condition and a thermal index (TI) condition be satisfied when the transmission beam is formed. That is, transmission power to send the transmission beam into the living body must be regulated so as to satisfy both the MI condition and the TI condition. In a case of forming the near focus wide beam, the sound pressure tends to be deficient in the divergent portion of the near focus wide beam. The sound pressure in the divergent portion can be raised by enhancing the transmission power, which will, however, increase sound energy concentrating on the focal point, resulting in a failure to satisfy the MI and TI conditions. Therefore, it is almost impossible to raise the sound pressure in the divergent portion of the near focus wide beam when the near focus wide beam having the single focal point is used.

JP 2003-175038 A (Patent Document 1) discloses a technique of forming a transmission beam. In this technique, a weighting addition is performed on two delay time curves. Patent Document 1 discloses no technique for combining a plurality of transmission beams in a living body.

JP 2015-192709 A (Patent Document 2) describes in FIG. 6 thereof that two transmission beams are simultaneously formed by setting two transmission apertures in a transducer array and setting two transmission focal points on a proximal end and a distal end of a focus area (FA). Patent Document 2 does not describe setting of the transmission focal point at a position shallower than a region of interest or utilization of a divergent portion of the transmission beams.

### SUMMARY

An object of the present disclosure is to form a transmission beam which is suitably spread in a region of interest. Another object of the present disclosure is to obtain a good sound pressure distribution in the region of interest while avoiding excessive concentration of sound energy within a living body.

An ultrasound diagnostic apparatus according to an aspect of this disclosure includes a transducer array and a controller configured to control operation of the transducer array, in which the operation of the transducer array is controlled to simultaneously form a plurality of transmission beams along a transmission center axis in such a manner that a plurality of transmission focal points are formed at a plurality of positions shallower than a region of interest on the transmission center axis, and a composite transmission beam is generated in a living body due to simultaneous formation of the plurality of transmission beams.

Abeam forming method according to another aspect of this disclosure includes setting a region of interest in a living body, defining a transmission condition for forming a plurality of transmission beams along a transmission center axis in such a manner that a plurality of transmission focal points are formed at a plurality of positions shallower than the region of interest on the transmission center axis, simultaneously forming the plurality of transmission beams according to the transmission condition, to generate a composite transmission beam within the living body, and simultaneously forming a plurality of reception beams after the composite transmission beam is formed.

### BRIEF DESCRIPTION OF DRAWINGS

Embodiments of the present disclosure will be described based on the following figures, wherein:

FIG. 1 is a block diagram showing an ultrasound diagnostic apparatus according to an embodiment;

FIG. 2 shows transmitting and receiving operation performed in a CFM mode;

FIG. 3 shows a single focus transmission beam and sound pressure distributions in the beam;

FIG. 4 shows a first example of a composite transmission beam according to the embodiment;

FIG. 5 shows a first comparative example;

FIG. 6 shows a second comparative example;

FIG. 7 shows a third comparative example;

FIG. 8 shows a second example of the composite transmission beam according to the embodiment;

FIG. 9 shows combinations of a plurality of focal depths;

FIG. 10 shows a first display example;

FIG. 11 shows a second display example;

FIG. 12 shows a flowchart showing an example of operation;

FIG. 13 shows a third example of the composite transmission beam according to the embodiment;

FIG. 14 shows a two-dimensional scan of the composite transmission beam;

FIG. 15 shows a first example of a transmission aperture pattern;

FIG. 16 shows a second example of the transmission aperture pattern;

FIG. 17 shows a third example of the transmission aperture pattern;

FIG. 18 shows a flowchart of a composite transmission beam designing method;

FIG. 19 shows target conditions;

FIG. 20 shows a plurality of transmission conditions; and

FIG. 21 shows results of evaluating the plurality of transmission conditions.

### DESCRIPTION OF EMBODIMENTS

Hereinafter, an embodiment is described with reference to the drawings.

### 1. Overview of Embodiment

An ultrasound diagnostic apparatus according to an embodiment includes a transducer array and a controller for controlling operation of the transducer array. The operation of the transducer array is controlled to simultaneously form a plurality of transmission beams along a transmission center axis in such a manner that a plurality of transmission focal points are formed at a plurality of positions shallower than a region of interest on the transmission center axis. As a result of simultaneous formation of the plurality of transmission beams, a composite transmission beam is formed in a living body.

According to the above-described configuration, because the plurality of transmission focal points are formed by a single transmission, concentration of sound energy on a single site within the living body can be prevented. In this way, enhancement of transmission power can be achieved, which can, in turn, allow a sound pressure in a divergent portion (a diffusing portion) to be raised. Further, a beam width of the composite transmission beam can be broadened in the region of interest, and in addition to the broadened beam width, a good sound distribution can be concurrently obtained in the region of interest. As a result, the blocky artifact hardly occurs when parallel reception is performed. The above-described configuration can also provide an advantageous effect that a form of the composite transmission beam can be changed relatively easily by individually changing conditions for forming the plurality of transmission beams.

Depths of the plurality of transmission focal points may be fixedly specified, or may be adaptively specified based on a depth of the region of interest (in particular, a depth of an upper edge or an upper surface of the region of interest).

In the embodiment, a portion of the composite transmission beam located in a region deeper than the plurality of focal points is a divergent portion. The divergent portion is designed to pass through the region of interest. The divergent portion has a two-dimensionally or three-dimensionally spreading form like a fan.

In the embodiment, the controller sets a plurality of transmission apertures in a transducer array. The plurality of transmission beams are simultaneously formed by the plurality of transmission apertures. In the embodiment, the plurality of transmission apertures include an inner transmission aperture and an outer transmission aperture established outward of the inner transmission aperture.

In the embodiment, the plurality of transmission beams include a first transmission beam formed by the inner transmission aperture and a second transmission beam formed by the outer transmission aperture. The plurality of transmission focal points include a first transmission focal point of the first transmission beam and a second transmission focal point of the second transmission beam. The first transmission focal point is a near focal point, and the second transmission focal point is a far focal point present at a position deeper than the near focal point. Alternatively, the first transmission focal point may be the far focal point, and the second focal point may be the near focal point present at a position shallower than the far focal point.

When the first transmission focal point formed by the inner transmission aperture is defined as the near focal point and the second transmission focal point formed by the outer transmission aperture is defined as the far focal point, the divergent portion having a moderately spreading shape can be formed. On the other hand, when the first transmission focal point formed by the inner transmission aperture is defined as the far focal point and the second transmission focal point formed by the outer transmission aperture is defined as the near focal point, the divergent portion having a significantly spreading shape can be formed.

The sound pressure distribution in the divergent portion and a beam width thereof can be arbitrarily manipulated by adjusting a first transmission beam forming condition and a second transmission beam forming condition, in particular, by adjusting the position of the first transmission focal point and the position of the second transmission focal point. For the purpose of suppressing a disturbance in a phase of a transmission wave reaching each observation point, it is preferable that an interval between the first transmission focal point and the second transmission focal point should not be broadened excessively.

In an embodiment, the region of interest is a three-dimensional region of interest. The transducer array is a two-dimensional transducer array. The inner transmission aperture is a two-dimensional transmission aperture. The outer transmission aperture is a two-dimensional transmission aperture defined to surround the inner transmission aperture. Each transmission beam is a three-dimensional transmission beam. The composite transmission beam is a three-dimensional composite transmission beam.

In the above-described configuration, because the beam width (specifically, a full width at half maximum in a first electronic scanning direction and a full width at half maximum in a second electronic scanning direction) in the divergent portion of the composite transmission beam can be increased, the blocky artifact hardly occurs when the parallel reception is performed. The three-dimensional transmission beam is a transmission beam which is formed by applying an electronic focusing technique to scanning in both the first electronic scanning direction and the second electronic scanning direction. It should be noted that the full width at half maximum (hereinafter abbreviated as FWHM) is usually defined as a width between two points located −6 dB below the peak of a sound pressure distribution on both sides of the peak.

In the embodiment, the controller controls formation of the plurality of transmission beams in accordance with a specific composite transmission beam forming condition selected from a plurality of composite transmission beam forming conditions. When the specific composite transmission beam forming condition is switched to another composite transmission beam forming condition, the sound pressure distribution in the composite transmission beam accordingly changes in the region of interest. A change in the sound pressure distribution includes a change in a beam width of the composite transmission beam. For example, the specific composite beam forming condition is selected automatically or manually based on the depth of the region of interest, a transmission beam density, and other factors.

In the embodiment, each of the composite transmission beam forming conditions includes a depth combination consisting of a plurality of transmission focal depths. A plurality of depth combinations defined in the plurality of transmission beam forming conditions differ from each other.

In the embodiment, the transducer array is configured to asynchronously form the single focus transmission beam and the composite transmission beam. The single focus transmission beam is a transmission beam used for acquiring tissue structure information. The composite transmission beam is a transmission beam used for acquiring tissue motion information. After the composite transmission beam is formed, a plurality of reception beams are simultaneously formed in order to acquire the tissue motion information. According to this configuration, both the tissue structure information and the tissue motion information can be acquired by appropriately using two types of transmission beams.

In the embodiment, the controller individually sets a depth of a transmission focal point of the single focus transmission beam and depths of a plurality of transmission focal points of the composite transmission beam. The ultrasound diagnostic apparatus according to the embodiment includes a generator for generating an image for a user to specify the depth of the transmission focal point of the single focus transmission beam and the depths of the plurality of transmission focal points of the composite transmission beam. A display processor, which will be described below, corresponds to the generator.

A beam forming method according to an embodiment includes a region of interest setting step, a transmission condition defining step, a transmitting step, and a receiving step. In the region of interest setting step, the region of interest is set within the living body. In the transmission condition defining step, a transmission condition for forming a plurality of transmission beams along the transmission center axis in such a manner that a plurality of transmission focal points are formed at a plurality of positions shallower than the region of interest on the transmission center axis is defined. In the transmitting step, the plurality of transmission beams are simultaneously formed in accordance with the transmission condition. As a result, the composite transmission beam is formed in the living body. In the receiving step, a plurality of reception beams are simultaneously formed after the composite transmission beam is formed.

According to the above-described method, a good sound pressure distribution can be obtained in the region of interest while preventing concentration of sound energy on a single point within the living body, which can, in turn, enhance image quality of an image representing the region of interest.

### 2. Details of Embodiment

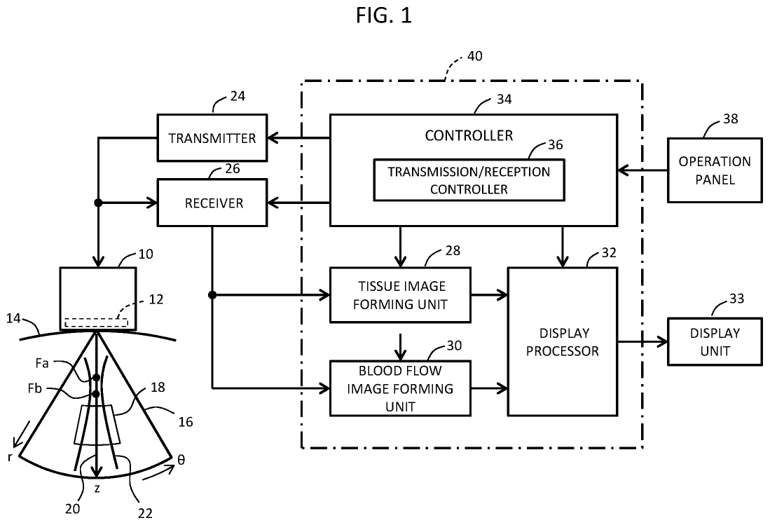

FIG. 1 shows an ultrasound diagnostic apparatus according to the embodiment. The ultrasound diagnostic apparatus is medical equipment used in medical institutions for conducting ultrasound examination.

A probe 10 includes a transducer array 12 composed of a plurality of transducers arranged in a straight line or a curved line. A transmitting/receiving surface of the probe 10 is brought into contact with a surface of a living body 14, and in this state, an ultrasound wave is transmitted into the living body 14. Then, a reflected wave generated inside the living body 14 is received.

Specifically, an ultrasound beam is formed by the transducer array 12, and the formed ultrasound beam is electronically scanned to form a scanning plane. In FIG. 1, a direction r represents a depth direction, and a direction θ represents an electronic scanning direction. As an electronic scanning mode, an electronic sector scan mode and an electronic linear scan mode, for example, have been known.

The ultrasound diagnostic apparatus according to the embodiment has a color flow mapping (CFM) mode. The CFM mode is also referred to as a color doppler mode. In the CFM mode, a first beam scanning plane for observing tissue structure and a second beam scanning plane for observing blood flow information are formed. In FIG. 1, reference numeral 16 represents the second beam scanning plane. For example, in accordance with a predetermined time division sequence, a plurality of transmission-receptions are performed to form the first beam scanning plane, and a plurality of transmission-receptions are performed to form the second beam scanning plane.

To form the first beam scanning plane, a transmission beam and a reception beam are sequentially formed in each azimuth direction. In actual operation, a plurality of reception beams are simultaneously formed (a reception beam set is formed) per one transmission beam in accordance with a parallel receive scheme. The one transmission beam has a single transmission focal point as in the case of a conventional transmission beam.

On the other hand, to form the second beam scanning plane 16, in each azimuth direction, a composite transmission beam 22 is formed, and a reception beam set is subsequently formed in accordance with the parallel receive scheme. In actual operation, a transmission beam having a near focal point Fa and a transmission beam having a far focal point Fb are formed at the same time, and are acoustically combined into the composite transmission beam 22 within the living body 14.

As will be described in detail below, both the near focal point Fa and the far focal point Fb are established on a transmission center axis 20 on a rear side (a probe 10 side) of a region of interest (ROI) 18. A divergent portion of the composite transmission beam 22 passes through the ROI 18. The ROI 18 is a blood flow observing region specified by a user who is a medical examiner (such as a doctor or a clinical technician).

The ROI 18 is, in the example illustrated in FIG. 1, a two-dimensional region having a fan shape or a trapezoidal shape. Typically, the transmission beam used for observing blood flow information; i.e., the composite transmission beam 22, is electronically scanned within a width, in the electronic scanning direction, of the ROI 18, and the reception beam set used for observing blood flow information is formed within the width of the ROI 18.

When three-dimensional color doppler imaging is performed, a probe equipped with a two-dimensional transducer array is used. The two-dimensional transducer array is composed of a plurality of transducers arranged in lines along a first direction and a second direction. As in the case of the above-described example, two transmission beams are simultaneously formed by the two-dimensional transducer array and combined in the living body into a composite transmission beam. The composite transmission beam is scanned along a first electronic scanning direction and a second electronic scanning direction. An electronic circuit for sub beam forming may be installed along with the two-dimensional transducer array in the probe. In this case, the electronic circuit may function as a transmitter 24 which is described below.

The transmitter 24 is an electronic circuit configured to supply, during transmission, a plurality of transmission signals in parallel to the transducer array 12, and functions as a transmission beamformer. A receiver 26 is an electronic circuit configured to perform, during reception, phase alignment and addition on a plurality of reception signals output in parallel from the transducer array 12, to form reception beams, and functions as a reception beamformer. In the receiver 26, data of a plurality of reception beams are generated in parallel in accordance with the parallel receive scheme. The receiver 26 includes a plurality of amplifiers, a plurality of A/D converters, a memory, an adder, and other components.

A plurality of sets of reception beam data acquired by forming the first scanning plane are transmitted to a tissue image forming unit 28. A plurality of sets of reception beam data acquired by forming the second scanning plane 16 are transmitted to a blood flow image forming unit 30.

The tissue image forming unit 28 generates a tomographic image (B mode tomographic image) representing tissue structure, based on the plurality of sets of reception beam data acquired by forming the first scanning plane. The tissue image forming unit 28 includes a beam data processor, a digital scan converter (DSC), and other components. The tissue image forming unit 28 may generate a three-dimensional tissue image. In this case, a plurality of sets of reception beam data (first volume data) acquired from a three-dimensional data acquisition region within the living body are supplied to the tissue image forming unit 28.

The blood flow image forming unit 30 generates a blood flow image (color doppler image) representing motion of a blood flow, based on the plurality of sets of reception beam data acquired by forming the second scanning plane 16. The blood flow image is, for example, an image representing a velocity distribution or an image representing a power distribution. The blood flow image forming unit 30 may generate an image representing the velocity distribution and a velocity dispersion distribution. The blood flow image forming unit 30 may generate, rather than the blood blow image, an image representing motion of a soft tissue.

The blood flow image forming unit 30 includes a clutter filter, an autocorrelator, a velocity calculator, and a DSC, for example. The blood flow image forming unit 30 may generate a three-dimensional blood flow image. In the case, a plurality of sets of reception beam data (second volume data) acquired from the three-dimensional data acquisition region within the living body are supplied to the blood flow image forming unit 30.

A display processor 32 has an image generating function, a color calculating function, and an image combining function, for example. In the CFM mode, the display processor 32 combines the tissue image and the blood flow image to generate a CFM image. In general, the tissue image is a monochrome image, and the blood flow image is a color image. The display processor 32 functions as a generator configured to generate the image used for selecting a composite beam forming condition, and functions as a generator configured to generate graphics which will be described further below.

The ultrasound image is displayed on a display unit 33. In the CFM mode, the display unit 33 displays the CFM image. The display unit 33 is composed of an organic EL display device or an LCD, for example. A three-dimensional CFM image generated based on both the three-dimensional tissue image and the three-dimensional blood blow image may be displayed on the display unit 33.

A controller 34 controls operation of each of the components illustrated in FIG. 1. The controller 34 has a transmission and reception controlling function. In FIG. 1, the transmission and reception controlling function is represented as a transmission/reception controller 36. The transmission/reception controller 36 controls the transmitter 24 and the receiver 26; that is, controls operation of the transducer array 12, to thereby control formation of the transmission beam and the reception beam.

In the embodiment, when the blood flow information is acquired in the CFM mode under control by the transmission/reception controller 36, two independent transmission apertures are set in the transducer array 12, and the first transmission beam and the second transmission beam are simultaneously formed by the two transmission apertures. As a result, the composite transmission beam is generated within the living body 14. The composite transmission beam may be formed when the issue image is generated.

The controller 34 is implemented by a processor configured to execute a program. The processor is composed of a CPU (Central Processing Unit), for example. An information processor 40 incorporating the controller 34 and other components may be implemented by a single processor or two or more processors. It should be noted that there have been known processors including an ASIC (Application Specific Integrated Circuit), an FPGA (Field Programmable Gate Array), and a GPU (Graphics Processing Unit), for example.

The controller 34 is connected to an operation panel 38. The operation panel 38 includes switches, knobs, a trackball, a keyboard, and other components. In the embodiment, the operation panel 38 is used by a user to set a region of interest, and to define or select a composite beam forming condition.

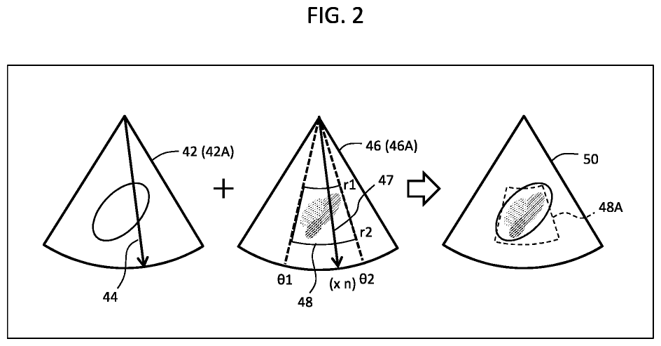

FIG. 2 shows transmitting and receiving operation in the CFM mode. A first scanning plane 42 is formed by repeating a transmitting and receiving step in the electronic scanning direction. Specifically, in each azimuth direction, a transmission beam 44 is formed and parallel reception is subsequently performed. Then, a tissue image (B mode tomographic image) 42A is generated based on frame data acquired from the first scanning plane 42.

A second scanning plane 46 is also formed by repeating a transmitting and receiving step in the electronic scanning direction. Here, n repetitions of the transmitting and receiving step are performed in each azimuth direction, where n is an integer greater than one, and may have a value from 4 to 16, for example. It should be noted that numerical values used herein are presented by way of illustration. In the embodiment, a composite transmission beam 47 is generated, and the parallel reception is subsequently performed in each transmitting and receiving step for acquiring the blood flow information.

In practice, the transmitting and receiving step is repeated in each azimuth direction within the width of a region of interest 48 specified by the user. The region of interest 48 is a fan shaped or trapezoidal region, and a top edge of the region of interest 48 is located at a depth r1, and a bottom edge of the region of interest 48 is located at a depth r2. The region of interest 48 has a width from θ1 to θ2 in the electronic scanning direction. The region of interest 48 corresponds to a blood flow observation area or a blood flow image display area. A blood flow image 46A is generated based on the frame data (specifically, doppler information) acquired from the second scanning plane 46.

A CFM image 50 is generated by overlaying the blood flow image 46A on the tissue image 42A. A reference sign 48A represents the region of interest. The CFM image 50 is a real time moving image representing motion of a heart and motion of a blood flow within the heart.

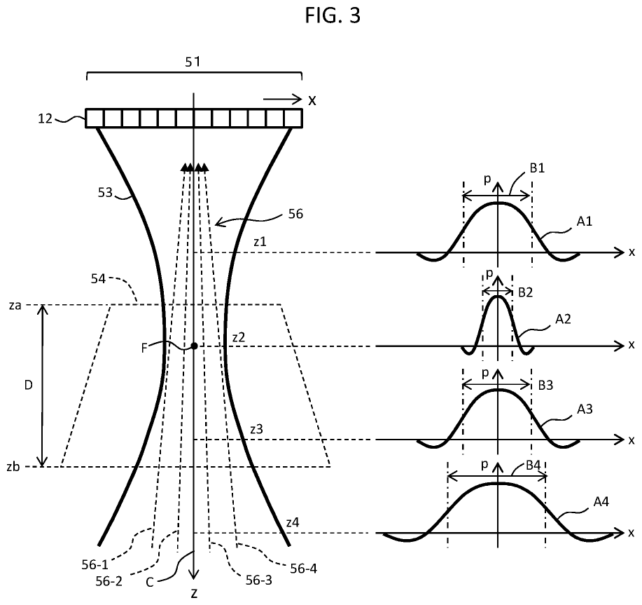

FIG. 3 shows a transmission beam 53 for forming a tissue image. An x direction shows a transducer arrangement direction. In the example shown in FIG. 3, a transmission and reception aperture 51 is set to the transducer array 12 in its entirety. The transmission and reception aperture 51 is used to form the transmission beam 53 and also form a reception beam set 56.

The transmission beam 53 has a single focal point F. In the example shown in FIG. 3, the focal point F is located within a region of interest 54. An upper end of the region of interest 54 is located at a depth za, and a lower end of the region of interest 54 is located at a depth zb. A range between the depths za and zb is a depth range D.

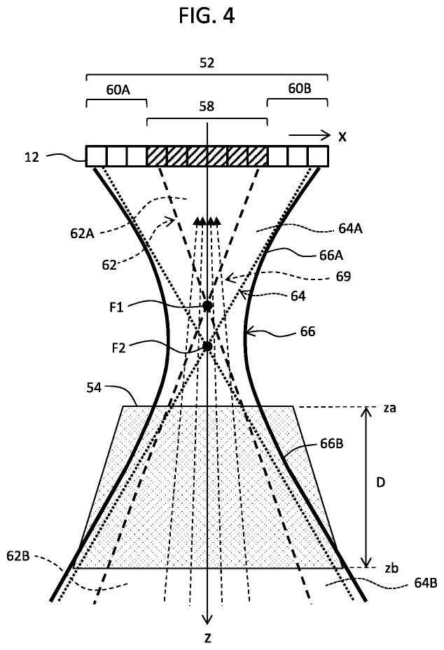

It should be noted that, in FIG. 3, the transmission beam 53 is schematically described in an exaggerated manner. This is also applied to FIGS. 4, 8, and 13 which will be referenced below. Further, in FIG. 3, the region of interest 54 is schematically represented for the purpose of reference, and in particular, the lateral width of the region of interest 54 is not precisely scaled, which is also applied to below-referenced FIGS. 4, 8, and 13.

FIG. 3 further shows sound pressure distribution curves A1 to A4 at depth positions z1 to z4 of the transmission beam 53. In each of the sound pressure distribution curves A1 to A4, the horizontal axis is an x axis being a spatial axis, and the vertical axis is a sound pressure axis (power axis). In the sound pressure distribution curves A1 to A4, a plurality of FWHMs B1 to B4 are indicated. In general, the FWHM corresponds to a distance between two points lower by −6 dB than the peak of the sound pressure distribution curve on both sides of the peak.

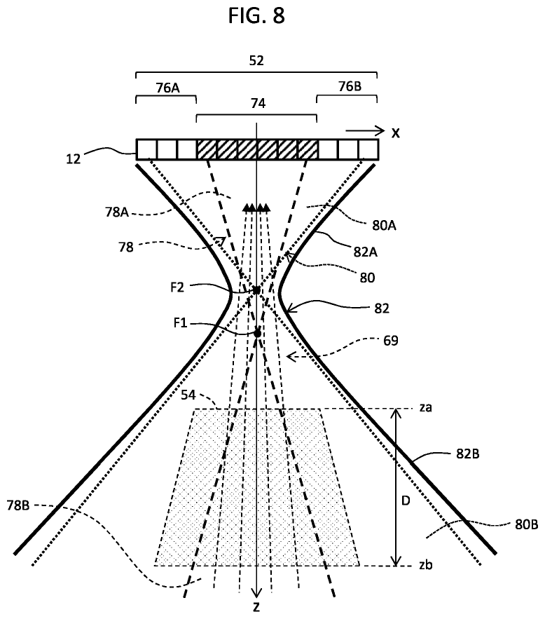

After the transmission beam 53 is formed, the reception beam set 56 consisting of reception beams 56-1 to 56-4 is formed in accordance with the parallel receive scheme. Although the reception beam set 56 typically includes 10 or more reception beams, for example, FIG. 3 schematically shows the reception beam set 56 consisting of a small number of reception beams 56-1 to 56-4. This is also applied to below-referenced FIGS. 4 and 8.

In a case of forming the tissue image, it is necessary that the tissue image have an increased quality over the entire area in the depth direction, while the blocky artifact is not particularly problematic. For this reason, the transmission beam 53 having the single transmission focal point F is used to generate the tissue image.

FIG. 4 shows a first example of a composite beam according to the embodiment. In the transducer array 12, an inner transmission aperture 58 is set and outer transmission apertures 60A and 60B are further set. In the illustrated example, the inner transmission aperture 58 is defined in a central region of the transducer array 12, and the outer transmission apertures 60A and 60B are respectively defined on both sides of the inner transmission aperture 58. Meanwhile, at the time of reception, a reception aperture 52 is defined in the transducer array 12 in its entirety.

A first transmission beam 62 is formed by means of the inner transmission aperture 58, and simultaneously with this, a second transmission beam 64 is formed by means of the outer transmission apertures 60A and 60B. The first transmission beam 62 has a first transmission focal point F1 located on a near side (a transducer array 12 side) of the region of interest 54 on the transmission center axis. The second transmission beam 64 has a second transmission focal point F2 located on the near side of the region of interest 54 on the transmission center axis. The first transmission focal point F1 is the near focal point, and the second transmission focal point F2 is the far focal point located at a position deeper than the first transmission focal point F1.

It should be noted that a relatively small distance is specified as an interval between the first transmission focal point Fa and the second transmission focal point Fb, in view of suppressing variations in phase among a plurality of transmission wave surfaces arriving at each observation point in the scanning plane. For example, the interval lies in a range of 3 to 10 mm. A lower limit of the range may be defined in view of preventing concentration of sound energy.

The first transmission beam 62 has a convergent portion (a focusing portion) 62A in a region on a near side of the first transmission focal point F1 and a divergent portion (a diffusing portion) 62B in a region on a far side of the first transmission focal point F1. Similarly, the second transmission beam 64 has a convergent portion 64A in a region on a near side of the second transmission focal point F2 and a divergent portion 64B in a region on a far side of the second transmission focal point F2.

As a result of simultaneous formation of the first transmission beam 62 and the second transmission beam 64, a composite transmission beam 66 is generated in the living body. The composite transmission beam 66 has a narrow portion in the vicinity of the two transmission focal points F1 and F2. The composite transmission beam 66 also has a convergent portion 66A in a region on a near side of the two transmission focal points F1 and F2 and a divergent portion 66B in a region on a far side of the two transmission focal points F1 and F2. The divergent portion 66B is spread out in the electronic scanning direction at each depth. In other words, the FWHM is suitably increased at each depth of the divergent portion 66B. The thus-formed divergent portion 66B passes through the region of interest 54.

After the composite transmission beam 66 is generated, a reception beam set 69 is formed in accordance with the parallel receive scheme. In the embodiment, electronic scanning of the divergent portion 62B is performed in the region of interest 54.

When the transmission beam density is low, because an interval between transmission beams is great, it is necessary that a width of the reception beam set be broadened in the electronic scanning direction. As opposed to this, the divergent portion 66B according to the embodiment passes through the region of interest 54, which can render the sound pressure distribution uniform to a certain extent over a broad area within the region of interest 54. Therefore, presence of a low sound pressure part in the reception beam set 69 within the region of interest 54 can be prevented. This can, in turn, effectively prevent occurrence of the blocky artifact in the blood flow image.

A shape of the divergent portion 66B and a sound pressure distribution therein can be arbitrarily manipulated by adjusting an aperture pattern, the depths of the two transmission focal points F1 and F2, or other parameters. Composite transmission beam forming conditions are optimized or selected based on a depth and a size of the region of interest, so as to appropriately form the divergent portion 66B.

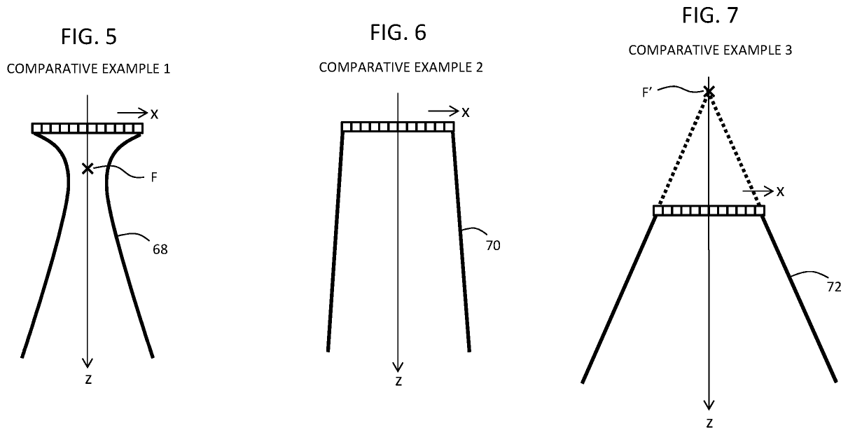

FIG. 5 shows a first comparative example. The first comparative example shows the use of a single focus, wide beam 68. In the first comparative example, when sound power is enhanced in order to raise a sound pressure in a divergent portion, sound energy will be excessively concentrated on the transmission focal point F. FIG. 6 shows a second comparative example, in which a planar wave 70 is transmitted. FIG. 7 shows a third comparative example, in which a divergent wave 72 is transmitted, taking a virtual transmission focal point F′ as a starting point. The second and third comparative examples present a problem in that the FWHM becomes excessively broad, resulting in a blurred blood flow image.

When the composite transmission beam according to the embodiment is used, sound power introduced into the living body can be enhanced; i.e., an average sound pressure can be raised in the divergent portion while preventing concentration of sound energy on one point. In addition, it becomes possible to broaden the FWHM as appropriate in the divergent portion.

FIG. 8 shows a second example of the composite transmission beam according to the embodiment. An inner transmission aperture 74 is set in the transducer array 12, and outer transmission apertures 76A and 76B are set on both sides of the inner transmission aperture 74. Further, at the time of reception, a reception aperture 52 is set in the entire area of the transducer array 12.

A first transmission beam 78 is formed using the inner transmission aperture 74, and simultaneously with this, a second transmission beam 80 is formed using the outer transmission apertures 76A and 76B. The first transmission beam 78 has the first transmission focal point F1 located on the near side of the region of interest 54 on the transmission center axis. The second transmission beam 80 has the second transmission focal point F2 located on the near side of the second transmission focal point F2 on the transmission center axis. As distinct from the first example, the first transmission focal point F1 is the far focal point, and the second transmission focal point F2 is the near focal point located at a position shallower than the first transmission focal point F1.

The first transmission beam 78 has a convergent portion 78A in the region on the near side of the first transmission focal point F1 and a divergent portion 78B in the region on the far side of the first transmission focal point F1. Similarly, the second transmission beam 80 has a convergent portion 80A in the region on the near side of the second transmission focal point F2, and a divergent portion 80B in the region on the far side of the second transmission focal point F2.

As a result of simultaneous formation of the first transmission beam 78 and the second transmission beam 80, a composite transmission beam 82 is generated in the living body. The composite transmission beam 82 has a convergent portion 82A in the region on the near side of the two transmission focal points F1 and F2, and a divergent portion 82B in the region on the far side of the two transmission focal points F1 and F2. The convergent portion 82A corresponds to a composition of the two convergent portions 78A and 80A. The divergent portion 82B corresponds to a composition of the two divergent portions 78B and 80B. The divergent portion 82B is spread out in the electronic scanning direction at each depth. In other words, the FWHM is increased at each depth of the divergent portion 82B. The thus-featured divergent portion 82B passes through the region of interest 54. After the formation of the composite transmission beam 82, a reception beam set 69 is formed in accordance with the parallel receive scheme.

Also in the second example, presence of the low sound pressure part in the reception beam set 69 within the region of interest 54 can be prevented. In this way, the presence of the above-described blocky artifact can be effectively prevented or reduced. Also in the second example, the shape of the divergent portion 66B and the sound pressure distribution therein can be manipulated by adjusting the aperture pattern, the depths of the two transmission focal points F1 and F2, or other factors.

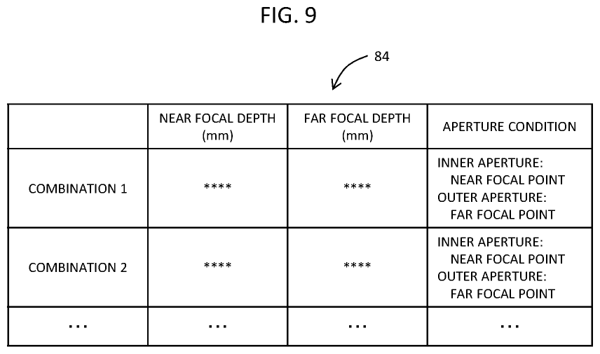

FIG. 9 shows a table 84 used for managing a plurality of focal depth combinations. Each of the focal depth combinations is defined with a near focal depth and a far focal depth, and also defined with aperture conditions. For example, the content of the table 84 or a list of the plurality of focal depth combinations is presented to the user. A specific focal depth combination is selected by the user. The controller sets in the transmitter a composite transmission beam forming condition that is fit for the selected focal depth combination. The composite transmission beam forming condition includes a transmission aperture condition, a delay condition, a transmission voltage condition, and a weighting condition, for example. When the composite transmission beam forming condition, in particular, the focal depth combination, is changed during operation in the CFM mode, the sound pressure distribution in the divergent portion of the composite transmission beam is accordingly changed, and in particular, the FWHM is changed. The composite transmission beam forming condition is selected based on a purpose of an examination, and an examination subject, for example.

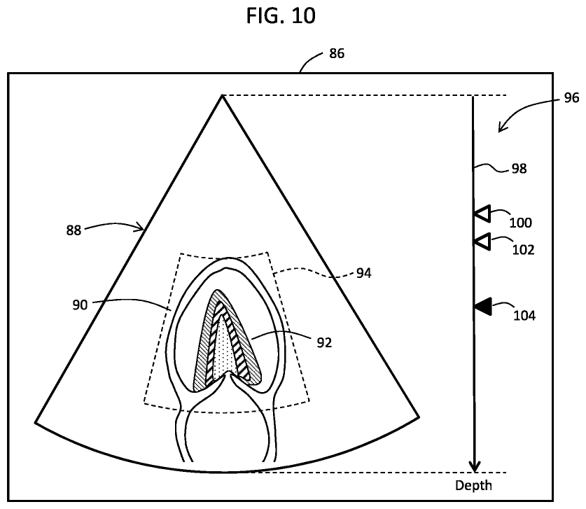

FIG. 10 shows a first display example. A display image 86 contains a CFM image 88. The CFM image 88 is composed of a tissue image 90 and a blood flow image 92. Reference numeral 94 represents a marker of the region of interest. Graphics 96 displayed along with the CFM image 88 include a depth axis 98 and a plurality of graphical items 100 to 104.

The graphical item 100 and the graphical item 102 are markers indicating the depths of the near focal point and the far focal point of the composite transmission beam for forming the blood flow image. The graphical item 104 is a marker indicating the depth of the transmission focal point of the transmission beam for forming a tissue image. The graphics 96 can allow the user to intuitively understand a depth relationship among the plurality of transmission focal points. Positions of the graphical items 100 to 104 may be designed to be slidable, for allowing the user to change each of the depths of the transmission focal points. The graphical items 100 to 104 have a triangular shape, while other forms of graphics may be used for the graphical items 100 to 104.

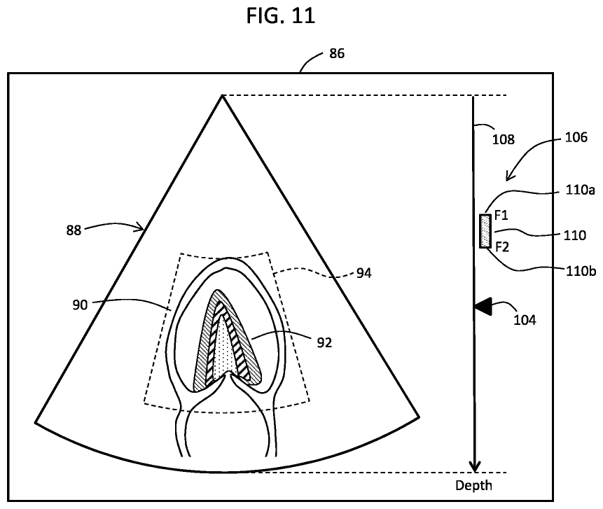

FIG. 11 shows a second display example. In FIG. 11, the same elements as those illustrated in FIG. 10 are designated by the same reference numerals as those illustrated in FIG. 10, and the descriptions related to the elements are not repeated. Graphics 106 include a depth axis 108 and the graphical items 110 and 104. An upper end 110a of the graphical item 110 indicates the depth of the near focal point. A lower end 110b of the graphical item 110 indicates the depth of the far focal point. The graphical item 110 has a rectangular shape in FIG. 11, while other forms of graphics may be used as the graphical item 110.

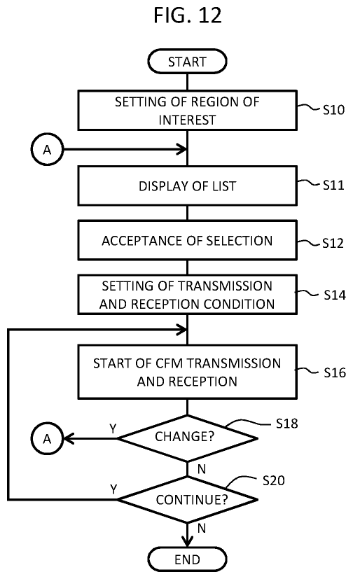

FIG. 12 shows a flowchart showing an example of operation in the CFM mode. In step S10, the region of interest is defined by the user, for example, on the tissue image. In step S11, the list of the composite transmission beam forming conditions is displayed, and in step S12 a specific composite transmission beam forming condition selected from the list by the user is accepted.

In step S14, an actual transmission condition is set in the transmitter based on the specific composite transmission beam forming condition, and an actual reception condition is set in the receiver. In step S16, transmissions and receptions according to the CFM mode are initiated. In step S18, when the composite transmission beam forming condition is changed in response to an input from the user or in response to automatic determination, operations in the steps from step S11 onward are performed again. The composite transmission beam forming condition may be changed until a desired degree of image quality is obtained; i.e., until a desired value of the HWHM and a desired sound pressure distribution are obtained. In step S20, it is determined whether or not to continue operation in the CFM mode.

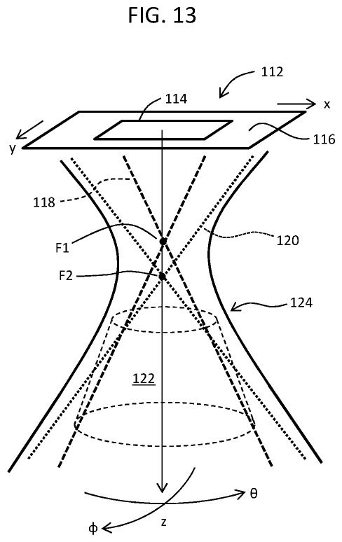

FIG. 13 shows a third example of the composite transmission beam according to the embodiment. A two-dimensional transducer array 112 is composed of a plurality of transducers arranged in lines along the x direction and the y direction. An inner transmission aperture 114 and an outer transmission aperture 116 are set in the two-dimensional transducer array 112. Specifically, the inner transmission aperture 114 is set in a central region of the two-dimensional transducer array 112, and the outer transmission aperture 116 is set so as to surround the inner transmission aperture 114. During reception, a reception aperture is set in the entire area of the two-dimensional transducer array 112.

A first transmission beam 118 is formed using the inner transmission aperture 114, and at the same time, a second transmission beam 120 is formed using the outer transmission aperture 116. The first transmission beam 118 has a first transmission focal point F1 located on a near side of a region of interest 122 on the transmission center axis. The second transmission beam 120 has a second transmission focal point F2 located on the near side of the region of interest 122 on the transmission center axis. The first transmission focal point F1 is the near focal point, and the second transmission focal point F2 is the far focal point. The near and far relationship between the first and second transmission focal points F1 and F2 may be reversed.

The first transmission beam 118 is a three-dimensional transmission beam formed by applying an electronic focusing technique to scanning in both a 0 direction (first electronic scanning direction) and a φ direction (second electronic scanning direction). Similarly, the second transmission beam 120 is also a three-dimensional transmission beam formed by applying the electronic focusing technique to scanning in both the 0 direction and the φ direction. The region of interest 122 is a three-dimensional region of interest extending in the depth direction, the θ direction, and the φ direction. The three-dimensional region of interest 122 has a conical or pyramid form. Alternatively, the three-dimensional region of interest 122 may have a cylindrical or prism form.

The first transmission beam 118 has a convergent portion in the region on the near side of the first transmission focal point F1 and a divergent portion in the region on the far side of the first transmission focal point F1. Similarly, the second transmission beam 102 has a convergent portion in the region on the near side of the second transmission focal point F2 and a divergent portion in the region on the far side of the second transmission focal point F2.

A composite transmission beam 124 is generated in the living body by simultaneously forming the first transmission beam 118 and the second transmission beam 120. The composite transmission beam 124 is a three-dimensional transmission beam. The composite transmission beam 124 has a convergent portion in the region on the near side of the two transmission focal points F1 and F2 and a divergent portion in the region on the far side of the two transmission focal points F1 and F2. The divergent portion extends at each depth in both the θ direction and the φ direction. The thus-formed divergent portion passes through the region of interest 122. After the formation of the composite transmission beam 124, a reception beam set composed of a plurality of reception beams which are aligned in the 0 direction and the φ direction is formed in accordance with the parallel receive scheme.

Also in the third example, the sound pressure distribution is rendered uniform to a certain extent over a broad area within the region of interest 122. Therefore, presence of the low sound pressure part in the reception beam set can be effectively prevented.

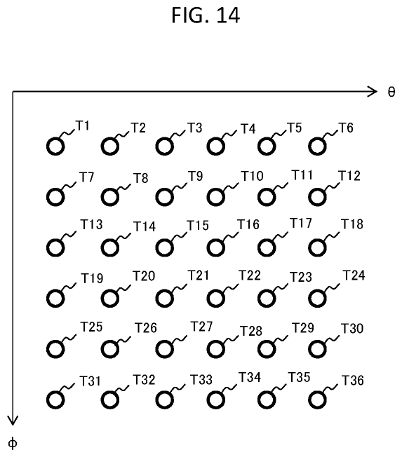

FIG. 14 shows an example of a transmission sequence employed to perform the three-dimensional color doppler imaging. The horizontal axis represents the θ direction, and the vertical axis represents the φ direction. Each graphical item indicates a transmission beam address; i.e., an azimuth of the transmission center axis. Reference letters T1 to T36 indicate ordinal number in the transmission sequence. In blood flow observation, the volume rate is required to have a value of, for example, 15 to 20 Hz or higher. To achieve this, the transmission beams aligned in the θ direction and the φ direction must be reduced in number, for example, to several transmission beams. Therefore, it is inevitable that the transmission beam density becomes significantly low when the three-dimensional color doppler imaging is performed. For this reason, it is necessary to increase the number of reception beams which are obtained per one transmitting and receiving step according to the parallel receive scheme.

In the above-described third example, because the divergent portion is spread in the two electronic scanning directions and a good sound pressure distribution can be obtained in the divergent portion, the occurrence of the low sound pressure part in the reception beam set can be prevented or suppressed even though the reception beam set is spatially expanded. This can cause the blocky artifact to hardly occur in imaging.

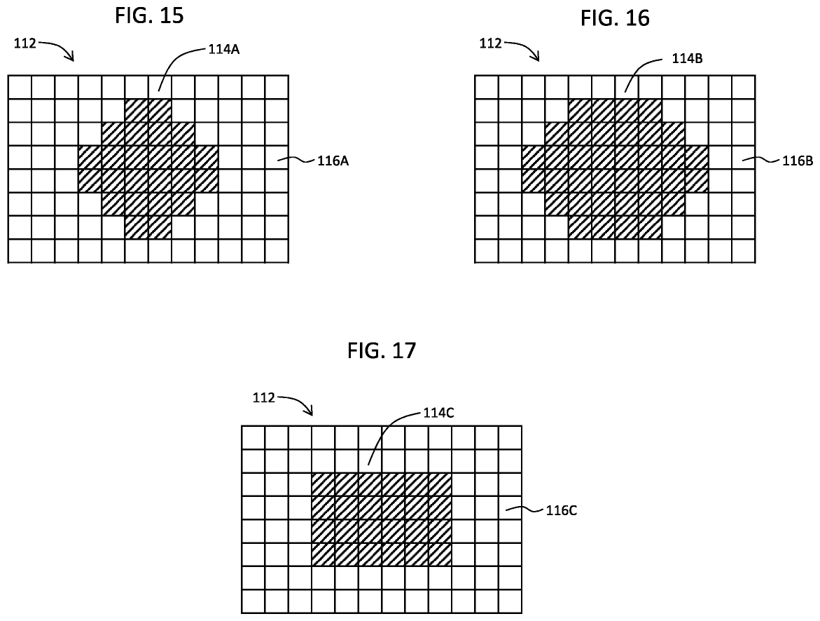

FIG. 15 shows a first example of a transmission aperture pattern which is applied to the two-dimensional transducer array 112. An inner transmission aperture 114A is defined in a shape approximating a circle, and an outer transmission aperture 116A is defined to surround the inner transmission aperture 114A.

FIG. 16 shows a second example of the transmission aperture pattern, in which an inner transmission aperture 114B is defined in a shape approximating an ellipse, and an outer transmission aperture 116B is defined to surround the inner transmission aperture 114B.

FIG. 17 shows a third example of the transmission aperture pattern, in which an inner transmission aperture 114C is defined in the shape of a rectangle, and an outer transmission aperture 116C is defined to surround the inner transmission aperture 114C. In the first to third examples, outer peripheral shapes of the outer transmission apertures may be formed in a circular or elliptical shape. Three or more transmission apertures may be provided to simultaneously form three or more transmission beams.

Next, a composite transmission beam designing method for realizing a desired FWHM and a desired sound pressure distribution in the divergent portion will be described.

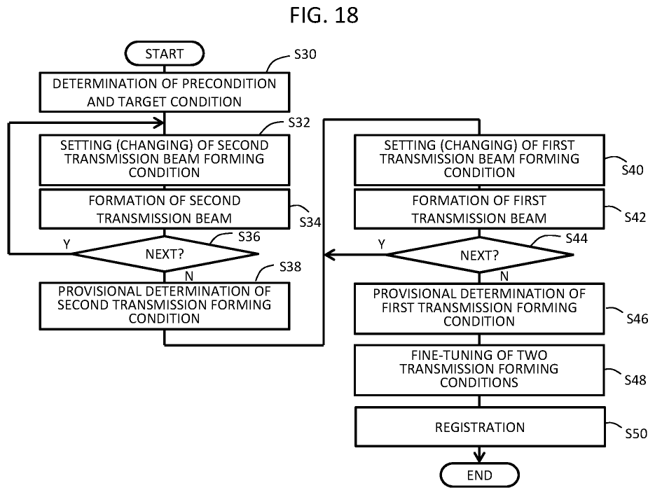

FIG. 18 shows an example of the composite transmission beam designing method in a flowchart. In step S30, a precondition and a target condition are determined. The precondition includes a position and a size of the region of interest, or a range of changes in the position and the size. The precondition further includes a transmission frequency, a transmission beam density, a wave number constituting a transmission pulse, a parallel receive condition, a probe type (type of a transducer array), an MI condition, a TI condition, and the like. The target condition includes a target value of the FWHM and a target sound pressure distribution for the divergent portion.

In step S32, a forming condition for forming the second transmission beam by the outer transmission aperture under the determined precondition is provisionally set. An outside shape of the divergent portion in the composite transmission beam is mainly defined by an outside shape of the divergent portion in the second transmission beam. It is therefore rational to start with the design of the second transmission beam.

In step S34, a trial second transmission beam is formed according to the provisionally set forming condition. The trial second transmission beam may be formed in a computer simulation. In step S36, the operations in steps S32 and S34 are repeated while changing forming conditions of the second transmission beam until it is determined that a form of the divergent portion that satisfies a fixed condition is obtained.

When it is determined in step S36 that a form of the divergent portion that satisfies a fixed condition is obtained, the forming condition of the second transmission beam yielding such a favorable result is provisionally defined as a usable second transmission beam forming condition in step S38. It should be noted that the fixed condition is defined in terms of the target condition.

In step S40, a forming condition for forming a first transmission beam with the inner transmission aperture under the precondition and the usable second transmission beam forming condition is provisionally set. In step S42, a trial first transmission beam is formed in accordance with the provisionally set forming condition. The trial first transmission beam may be formed in a computer simulation. In step S44, the operations in steps S40 and S42 are repeated while changing forming conditions of the first transmission beam until it is determined that a form of the divergent portion that satisfies the target condition is obtained in the composite transmission beam.

When it is determined in step S44 that a form of the divergent portion that satisfies the target condition is obtained in the composite transmission beam, the forming condition of the first transmission beam yielding such a favorable result is provisionally set as a usable first transmission beam forming condition in step S46. In evaluation performed in step S44, in particular, the degree of side lobe cancellation between the first transmission beam and the second transmission beam in the region of interest is evaluated.

In step S48, the usable first transmission beam forming condition and the usable second transmission beam forming condition are fine-tuned as needed. In step S50, the fine-tuned forming conditions are finalized and registered as the first transmission beam forming condition and the second transmission beam forming condition. Each of the forming conditions includes an aperture condition and a transmission focal depth, and other parameters.

Hereinafter, mathematical expressions which can be used to determine the transmission beam forming conditions are explained for reference purposes. A target condition of the FWHM is described, for example, by Expression (1) as follows.

[Expression 1]

αd<Hₑ (1)

In Expression (1), He represents the FWHM of a transmission beam, d represents a pitch between transmission beams, and α represents a predetermined coefficient. As a value of the FWHM, a representative value (such as an average value, a maximum value, or a minimum value) of FWHMs in the region of interest can be used.

A condition that a representative sound pressure in the region of interest exceeds a predetermined threshold value may be used as a sound pressure condition. The representative sound pressure may include an average sound pressure expressed on the left side of below-described Expression (2) and a sound pressure in the deepest part expressed on the right side of below-described Expression (3).

$$\begin{matrix}[ Expression 2 ] & \\ \frac{{\int }^{R O I} P dV}{{V}_{ROI}} > {P}_{s 1} & ( 2 )\end{matrix}$$

$$\begin{matrix}[ Expression 3 ] & \\ {P}_{z} > {P}_{s2} & ( 3 )\end{matrix}$$

In above Expressions 2 and 3, P represents a sound pressure distribution, V represents a volume, ROI represents the region of interest, V_{ROI} represents a volume of the region of interest, and Pz represents a sound pressure at the deepest position in the region of interest. Further, Ps1 and Ps2 represent threshold values.

There may be employed a condition that a sound pressure at a maximum depth in a round-trip region of the ultrasound pulse exceeds a predetermined threshold value. Optimum conditional expressions and optimum threshold values may be found by previously conducting a numerically simulated imaging or actual imaging and evaluating the extent to which the blocky artifact occurs in imaging.

The relationship between a volume rate and a transmission beam pitch can be described by Expression (4) as follows.

$$\begin{matrix}[ Expression 4 ] & \\ d = θ \sqrt{\frac{N}{PRF} {( \frac{1}{VR} - {t}_{B} )}^{- 1}} & ( 4 )\end{matrix}$$

In Expression (4), VR represents the volume rate, PRF represents a pulse repetition frequency, t_{B} represents a length of time required for taking a B mode image, N represents the number of repetitive transmissions for color doppler imaging (corresponding to n shown in FIG. 2), and θ represents an angle of view. Expression (4) is created based on the premise that the number of transmission beams arranged in the first electronic scanning direction is equal to the number of transmission beams arranged in the second electronic scanning direction.

In the three-dimensional color doppler imaging, because the transmission beam is two-dimensionally scanned, when the volume rate is doubled, the transmission beam pitch is increased approximately in proportion to the square root of the volume rate. Based on this relationship and Expression (1), below-described Expression (5) can be derived as a conditional expression.

$$\begin{matrix}[ Expression 5 ] & \\ {H}_{e} > α θ \sqrt{\frac{N}{PRF} {( \frac{1}{VR} - {t}_{B} )}^{- 1}} & ( 5 )\end{matrix}$$

On the other hand, in the two-dimensional color doppler imaging, because the transmission bean is one-dimensionally scanned, the transmission beam pitch is approximately proportional to the frame rate. As a conditional expression in this case, below-described Expression (6) can be used.

$$\begin{matrix}[ Expression 6 ] & \\ {H}_{e} > α θ \sqrt{\frac{N}{PRF} {( \frac{1}{FR} - {t}_{B} )}^{- 1}} & ( 6 )\end{matrix}$$

In Expression (6), FR represents the frame rate.

A transmission delay time applied to each transmission element can be calculated by Expression (7) as follows.

$$\begin{matrix}[ Expression 7 ] & \\ τ = \frac{\sqrt{{( {x}_{f} - {x}_{i} )}^{2} + {( {y}_{f} - {y}_{i} )}^{2} + {z}_{{f}^{2}}}}{C} & ( 7 )\end{matrix}$$

In Expression (7), xᵢ and yᵢ represent x and y coordinates of an i^{th} transmission element, x_{f}, y_{f}, and z_{f} represent coordinates of the transmission focal point specified to the i^{th} transmission element, τ represents a transmission delay time, and c represents a sound velocity in the living body. A smoothing filter may be applied to a plurality of transmission delay times calculated by Expression (7) for a plurality of transmission elements.

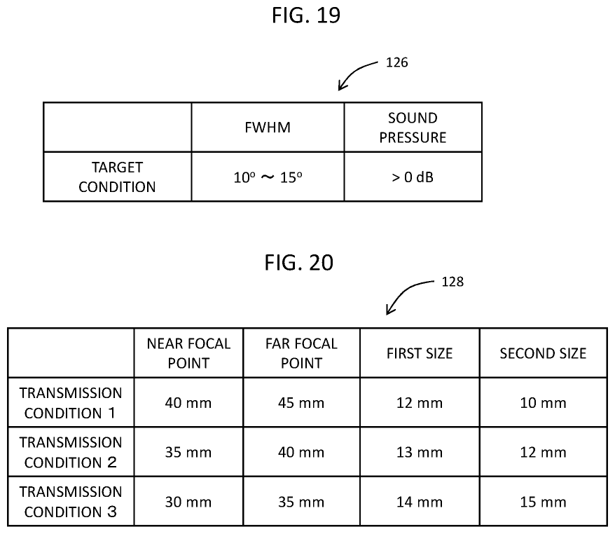

FIG. 19 shows an example of the target condition. A target condition 126 illustrated in FIG. 19 includes conditions of the FWHM and the sound pressure. The composite transmission beam is designed to satisfy the target condition 126.

FIG. 20 shows a list 128 of a plurality of transmission conditions (composite transmission beam forming conditions) generated by implementing the composite transmission beam designing method. Each of the transmission conditions includes the near focal depth, the far focal depth, a first size (size in the first electronic scanning direction) of the inner transmission aperture, and a second size (size in the second electronic scanning direction) of the inner transmission aperture.

Prior to performing imaging in the CFM mode, the list shown in FIG. 20 may be presented to the user, and a specific transmission condition (specific composite transmission beam forming condition) may be selected from the list by the user. The transmission condition may be switched another transmission condition during the imaging in the CFM mode.

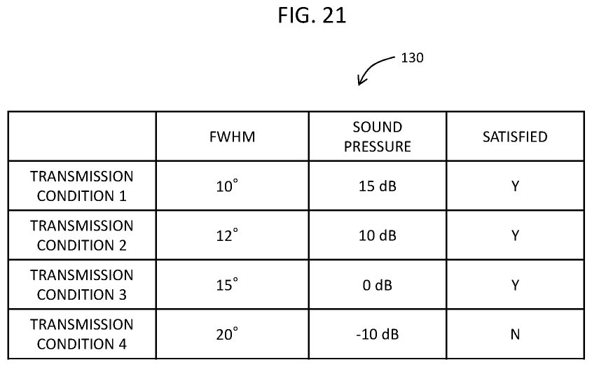

An evaluation result table 130 shown in FIG. 21 may be presented to the user. The evaluation result table 130 may be displayed along with the above-described list or may be displayed independently of the list. The evaluation result table 130 indicates evaluation results on a transmission condition by transmission condition basis.

Specifically, the evaluation result table 130 includes values of the FWHM (representative FWHM) and the sound pressure (representative sound pressure) for each of the transmission conditions, and results of determination as to whether or not the target condition is satisfied under each of the transmission conditions. The evaluation result table 130 illustrated in FIG. 21 shows a transmission condition 4 which does not satisfy the targe condition. When the list illustrated in FIG. 20 is displayed, the transmission condition 4 may be excluded from the list.

As has been described above, according to the embodiment, the good sound pressure distribution can be obtained in the region of interest while preventing excessive concentration of sound energy within the living body. As a result, occurrence of the blocky artifact in imaging in the CFM mode can be effectively suppressed, and, in particular, imaging in the three-dimensional CFM mode can be achieved, which can, in turn, improve quality of the two-dimensional or three-dimensional blood flow image.

1. An ultrasound diagnostic apparatus, comprising:
a transducer array; and
a controller configured to control operation of the transducer array; wherein
the operation of the transducer array is controlled to simultaneously form a plurality of transmission beams along a transmission center axis in such a manner that a plurality of transmission focal points are formed at a plurality of positions shallower than a region of interest on the transmission center axis, and
a composite transmission beam is generated in a living body due to simultaneous formation of the plurality of transmission beams.

2. The ultrasound diagnostic apparatus according to claim 1, wherein:
the composite transmission beam has a divergent portion in a region deeper than the plurality of transmission focal points; and
the divergent portion passes through the region of interest.

3. The ultrasound diagnostic apparatus according to claim 1, wherein:
the controller is further configured to set a plurality of transmission apertures in the transducer array, and
the plurality of transmission beams are simultaneously formed by the plurality of transmission apertures.

4. The ultrasound diagnostic apparatus according to claim 3, wherein:
the plurality of transmission apertures comprise
an inner transmission aperture, and
an outer transmission aperture defined outside the inner transmission aperture.

5. The ultrasound diagnostic apparatus according to claim 4, wherein:
the plurality of transmission beams comprise
a first transmission beam formed by the inner transmission aperture, and
a second transmission beam formed by the outer transmission aperture;
the plurality of transmission focal points comprise
a first transmission focal point of the first transmission beam, and
a second transmission focal point of the second transmission beam;
the first transmission focal point is a near focal point; and
the second transmission focal point is a far focal point present at a position deeper than the near focal point.

6. The ultrasound diagnostic apparatus according to claim 4, wherein:
the plurality of transmission beams comprise
a first transmission beam formed by the inner transmission aperture, and
a second transmission beam formed by the outer transmission aperture;
the plurality of transmission focal points comprise
a first transmission focal point of the first transmission beam, and
a second transmission focal point of the second transmission beam;
the first transmission focal point is a far focal point; and
the second transmission focal point is a near focal point present at a position shallower than the far focal point.

7. The ultrasound diagnostic apparatus according to claim 4, wherein:
the region of interest is a three-dimensional region of interest;
the transducer array is a two-dimensional transducer array;
the inner transmission aperture is a two-dimensional transmission aperture;
the outer transmission aperture is a two dimensional transmission aperture defined to surround the inner transmission aperture;
each of the transmission beams is a three-dimensional transmission beam; and
the composite transmission beam is a three-dimensional composite transmission beam.

8. The ultrasound diagnostic apparatus according to claim 1, wherein:
the controller is configured to control formation of the plurality of transmission beams according to a specific composite transmission beam forming condition selected from a plurality of composite transmission beam forming conditions;
a sound pressure distribution in the composite transmission beam is changed within the region of interest by switching the specific composite transmission beam forming condition to another one of the composite transmission beam forming conditions; and
a change in the sound pressure distribution comprises a change in a beam width of the composite transmission beam.

9. The ultrasound diagnostic apparatus according to claim 8, wherein:
each of the plurality of composite transmission forming conditions comprises a depth combination of a plurality of transmission focal depths; and
a plurality of depth combinations in the plurality of composite transmission beam forming conditions differ from each other.

10. The ultrasound diagnostic apparatus according to claim 1, wherein:
the transducer array is configured to asynchronously form a single focus transmission beam and the composite transmission beam;
the single focus transmission beam is a transmission beam for acquiring tissue structure information;
the composite transmission beam is a transmission beam for acquiring tissue motion information; and
a plurality of reception beams are simultaneously generated to acquire the tissue motion information after the composite transmission beam is formed.

11. The ultrasound diagnostic apparatus according to claim 10, wherein
the controller is further configured to individually set a transmission focal depth of the single focus transmission beam, and the plurality of transmission focal depths of the composite transmission beam.

12. The ultrasound diagnostic apparatus according to claim 11, further comprising:
a generator configured to generate an image for a user to specify the transmission focal depth of the single focus transmission beam and the plurality of transmission focal depths of the composite transmission beam.

13. A beam forming method, comprising:
defining a region of interest in a living body;
defining a transmission condition for forming a plurality of transmission beams along a transmission center axis in such a manner that a plurality of transmission formal points are formed at a plurality of positions shallower than the region of interest on the transmission center axis;
simultaneously forming the plurality of transmission beams according to the transmission condition, to generate in the living body a composite transmission beam; and
simultaneously forming a plurality of reception beams after the composite transmission beam is formed.
